(19) United States
(12) Patent Application Publication
(10) Pub. No.: US 2013/0108552 A1
(43) Pub. Date: May 2, 2013

(54) NEAR-IR INDOCYANINE GREEN DOPED MULTIMODAL SILICA NANOPARTICLES AND METHODS FOR MAKING THE SAME

(71) Applicants: Parvesh Sharma, Gainesville, FL (US); Scott Chang Brown, Hockessin, DE (US); Niclas Bengtsson, Lake Forest Park, WA (US); Glenn A. Walter, Newberry, FL (US); Nobutaka Iwakuma, Kurume (JP); Edward W. Scott, Gainesville, FL (US); Stephen R. Grobmyer, Gainesville, FL (US); Swadeshmukul Santra, Orlando, FL (US); Brij M. Moudgil, Gainesville, FL (US)

(72) Inventors: Parvesh Sharma, Gainesville, FL (US); Scott Chang Brown, Hockessin, DE (US); Niclas Bengtsson, Lake Forest Park, WA (US); Glenn A. Walter, Newberry, FL (US); Nobutaka Iwakuma, Kurume (JP); Edward W. Scott, Gainesville, FL (US); Stephen R. Grobmyer, Gainesville, FL (US); Swadeshmukul Santra, Orlando, FL (US); Brij M. Moudgil, Gainesville, FL (US)

(73) Assignee: UNIVERSITY OF FLORIDA RESEARCH FOUNDATION INC., GAINESVILLE, FL (US)

(21) Appl. No.: 13/582,226

(22) PCT Filed: Feb. 24, 2011
(86) PCT No.: PCT/US2011/026038

§ 371 Date: Jan. 17, 2013

Related U.S. Application Data
(60) Provisional application No. 61/309,261, filed on Mar. 1, 2010.

(51) Int. Cl. A61N 5/06; A61K 49/00; G01N 21/64

(52) U.S. Cl. CPC A61N 5/062; G01N 21/6486; A61K 49/001
USPC 424/9.32; 436/172; 424/9.6; 424/490; 424/9.3; 604/20

(57) ABSTRACT

The subject invention provides novel fluorescent core-shell nanoparticles comprising an encapsulated fluorescent core comprising an ionically bound fluorescent dye and a metal oxide shell. In one exemplary embodiment of the invention a core containing indocyanine green (ICG) with a silica shell that displays excellent photostability for generation of a near infrared fluorescence signal. The fluorescent core-shell nanoparticle can be further modified to act as an MRI, x-ray, or PAT contrast agent. The ICG nanoparticles can also be used as photodynamic therapeutic agent. Other embodiments of the invention directed to methods of making the novel core-shell nanoparticles and to the use of the core-shell nanoparticles for in vitro or in vivo imaging.

### CROSS-REFERENCE TO RELATED APPLICATION

The present application claims the benefit of U.S. Provisional Application Ser. No. 61/309,261, filed Mar. 1, 2010, the disclosure of which is hereby incorporated by reference in its entirety, including any figures, tables, or drawings.

The subject invention was made with government support under the National Science Foundation, Contract No. EEC0506560. The government has certain rights to this invention.

### BACKGROUND OF THE INVENTION

Fluorescent dyes are widely used for near-infrared imaging but many applications of these dyes are limited by disadvantageous properties in aqueous solution that include concentration-dependent aggregation, poor aqueous stability in vitro and low quantum yield. For example, a particularly useful and FDA approved dye, indocyanine green (ICG), is known to strongly bind to nonspecific plasma proteins, leading to rapid elimination from the body, having a half-life of only 3-4 min. Other limiting factors displayed by ICG include: rapid circulation kinetics; lack of target specificity; and changes in optical properties due to influences such as concentration, solvent, pH, and temperature. To overcome some of these shortcomings the inclusion of the fluorescent dyes into micellar and nanoparticulate systems have been examined.

Attempts to encapsulate ICG into silica and polymer matrices have been met with only partial success. Much of this appears to stem from ICG's combined amphiphilic character and strong hydrophilicity. It contains both lipophilic groups and hydrophilic groups that promote its distribution at interfaces and its interaction with the surfactants that are often necessitated in the particles synthesis and largely limits its incorporation to the interior of nanoparticles. ICG displays a critical micelle concentration of about 0.32 mg/mL in H₂O and readily partitions into aqueous environments, and, therefore, ICG encapsulation in particulate matrices suffers from significant leaching.

Nevertheless, encapsulated ICG and other fluorescent dyes remain attractive for bio-imaging techniques that non-invasively measure biological functions, evaluate cellular and molecular events, and reveal the inner mechanisms of a body. Fluorescent dye comprising nanoparticles are useful for in vitro fluorescence microscopy and flow cytometry. Additionally, fluorescent dye comprising nanoparticles are potentially valuable for photoacoustic tomography (PAT), an emerging non-invasive in vivo imaging modality that uses a non-ionizing optical (pulsed laser) source to generate contrast. A PAT signal is detected as an acoustic signal whose scattering is 2-3 orders of magnitude weaker than optical scattering in biological tissues, a primary limitation of optical imaging.

Additionally, diagnosis often necessitates the use of more than one imaging technique to integrate the strengths of multiple techniques and overcome the limitations of an individual technique to improve diagnostics, preclinical research and therapeutic monitoring. Examples of PAT complementary techniques include magnetic resonance imaging (MRI), positron emission tomography (PET), X-ray tomography, luminescence (optical imaging), and ultrasound. Typically, analysis by different techniques requires different contrast agents. Furthermore, using multiple bio-imaging techniques requires significantly greater time and expense, and can impose diagnostic complications. If the fluorescent dye comprising nanoparticles include one or more additional contrast agents, multiple bio-imaging techniques could be carried out rapidly or simultaneously. Multi-modal contrast bio-imaging agents are potentially important tools for developing and benchmarking experimental imaging technologies by carrying out parallel experiments using developing and proven techniques.

To these ends, effective and stable fluorescent dye comprising nanoparticles and methods for their preparation are needed. Such novel nanoparticles could be employed for multiple biological applications, including imaging, even multiple bio-imaging techniques, and therapeutics.

### BRIEF SUMMARY OF THE INVENTION

Embodiments of the invention are directed to fluorescent core-shell nanoparticle wherein a core comprising a water soluble fluorescent dye is encapsulated in a silica shell. The dye is ion-paired with a cationic polymer and/or with a multivalent cation as a precipitated non-soluble matrix. In an exemplary embodiment of the subject invention, a FDA approved fluorescent dye, indocyanine green (ICG), is used. In one embodiment, the cationic polymer is chitosan treated by tripolyphosphate. In another embodiment, the multivalent cation is Ba²⁺ and the dye is distributed in precipitated BaSO₄. The novel core-shell nanoparticles can be monodispersed with sizes less than 100 nm.

Embodiments of the invention are directed to methods of making the novel fluorescent core-shell nanoparticle. This is done by using a water-in-oil microemulsion directed synthesis. In one embodiment, the preparation steps comprise: providing core within the water phase of a water-in-oil microemulsion where the core comprises a polymer having cationic sites, such as protonated chitosan, and/or an insoluble salt of a multivalent cation, such as a Ba²⁺ salt with a fluorescent dye having a plurality of anionic sites, such as ICG, and coating the core with a metal oxide layer, for example a silica layer, by condensation of a precursor, for example, ammonium carbonate catalyzed condensation of silanes.

Advantageously, fluorescent core-shell nanoparticles according to embodiments of the invention display good photostability. The synthetic methods used for the novel core-shell nanoparticle allow a multistep architecture on the nanoparticle, where, for example, the use of barium sulfate enables CT or X-ray contrast as well as near infrared fluorescence traceability and/or the inclusion of other contrast agents for robust multimodal bioimaging.

### BRIEF DESCRIPTION OF THE DRAWINGS

FIG. 1 is a schematic representation of chitosan stabilized indocyanine green (ICG) dye encapsulated in the silica matrix coated with polyethylene glycol (PEG) according to an embodiment of the invention.

FIG. 2 is a schematic illustration of the ionic interaction between bivalent cation Ba²⁺ and the sulfonate groups of single ICG dianion.

FIG. 3 shows (left) a TEM picture with a scale bar indicating 50 nm for about 25 nm ICG-BaSO₄ silica nanoparticles according to an embodiment of the invention and (right) an energy dispersive X-Ray spectrum that indicates the constituent elements of the ICG-BaSO₄ nanoparticles.

FIG. 4 shows a visible fluorescence microscopy image (×60) of washed BT474 cells after exposure to ICG core-shell nanoparticles for 24 hours according to an embodiment of the invention where the ICG core-shell nanoparticles appear red (bright) with blue nuclear staining from Hoechst 33258.

FIG. 5 shows photoacoustic images using ICG core-shell nanoparticles according to an embodiment of the invention in (a) tissue like phantom at depth of 1 cm for a 3 μL injection of 3 mg/mL suspension and (b) following an intratumoral injection of 10 μL of a 3 mg/mL suspension into a mouse bearing human breast tumor.

FIG. 6 shows photostability of 20 nm (displayed in a TEM image in an inset) ICG-BaSO₄-aminated silica core-shell nanoparticles according to an embodiment of the invention versus free ICG dye on continuous illumination.

FIG. 7 shows photobleaching of ICG core-shell nanoparticles according to an embodiment of the invention and ICG dye on continuous illumination.

FIG. 8 shows fluorescence from (A) ICG core-shell nanoparticles according to an embodiment of the invention obtained after centrifugation and re-dispersion in water; (B) supernatant and (C) ICG dye on continuous illumination.

FIG. 9 shows increased photostability of the ICG core-shell nanoparticles according to an embodiment of the invention as compared to ICG dye.

FIG. 10 shows the fluorescence emission spectra of ICG core-shell nanoparticles according to an embodiment of the invention and ICG dye with maxima at 800 nm (710 nm excitation).

FIG. 11 shows the fluorescence emission spectra of the ICG core-shell nanoparticles (dual emission) according to an embodiment of the invention and ICG dye upon excitation at 475 nm.

FIG. 12 shows visible light fluorescence from multimodal ICG-Gd core-shell nanoparticles labeled J-774 macrophage cells according to an embodiment of the invention.

FIG. 13 shows multiple fluorescence microscopy images of ICG core shell nanoparticle decorated breast cancer cells using three filter settings: Alexa 488, Alexa 633 and Alexa 750 according to an embodiment of the invention.

FIG. 14 shows NIR fluorescence (745 nm Excitation; 820 nm Emission) from multimodal ICG-Gd core-shell nanoparticles labeled cells according to an embodiment of the invention.

FIG. 15 shows MR contrast generated in cells using ICG-Gd core-shell nanoparticles according to an embodiment of the invention, where the labeled cells can be imaged by T1 (left) and T2 (right) weighted sequences.

FIG. 16 shows (left) real-time imaging using nude mice where tail vein had been injected with ICG core-shell nanoparticles after 60 minutes according to an embodiment of the invention and (right) monitored for over 150 minutes.

### DETAILED DISCLOSURE OF THE INVENTION

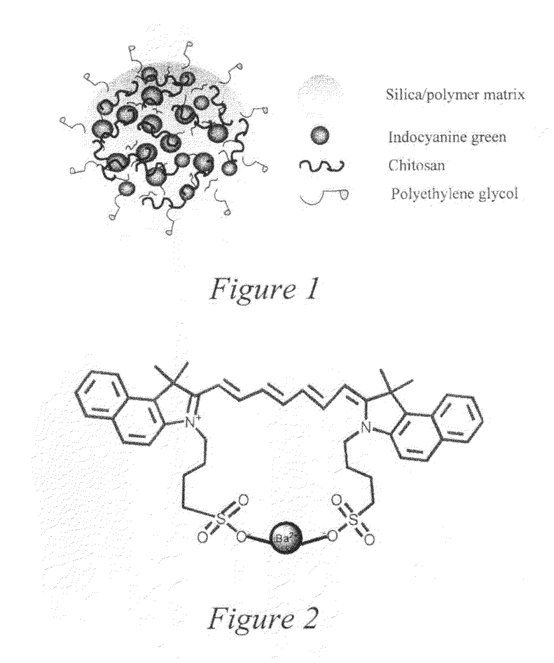

Embodiments of the invention are directed to fluorescent core-shell nanoparticles containing ionically bound ICG or other fluorescent dyes where the dye has at least one anionic site and is included within a core bound within an insoluble difunctional or multifunctional metal salt or ionically bound to a biocompatible polymer having a plurality of cationic sites and crosslinked into an insoluble polymer matrix core, and where the core is encapsulated in a metal oxide shell. Other fluorescent dyes that can be use in place of or in addition to ICG include, but are not limited to, Evans blue, bromothymol blue, and rose Bengal. For purposes of the invention, the core is a material that is formed in a first step and the shell is a material that is formed in a second step, and although in many embodiments of the invention the shell material will have limited penetration into the core material, in some embodiments of the invention, the shell material can penetrate deeply into or extending throughout the core material, yet the core and shell materials remain separate material phases. A simplified schematic representation of the particle design is shown in FIG. 1, where multiple core particles are dispersed within a metal oxide (silica) matrix with silica at the surface of the matrix. By including appropriate insoluble salts, the fluorescent nanoparticles can display X-ray, CT, and/or MRI contrast properties in addition to the fluorescence properties. Insoluble salts include, but are not limited to, barium sulfate, calcium oxalates, calcium fluoride, and ferric orthophosphate. In other embodiments of the invention, the nanoparticle can be further decorated to include aptamers, metal speckles, and/or groups to enhance solubility, affinity, or resistance to absorption or agglomeration of the fluorescent nanoparticles for use in a desired environment, for example in vivo. The ICG or other fluorescent dyes can be fixed within the fluorescent nanoparticles in a manner such that the dye can leach into a tumor or other structure and used as a therapeutic agent.

Some embodiments of the invention are directed to a method of preparing the novel fluorescent core-shell nanoparticles. The method involves formation of a core by a water-in-oil microemulsion directed synthesis. The oil can be any water immiscible liquid, for example a hydrocarbon such as hexane, cyclohexane, heptane, or iso-octane. The size of the nanoparticle cores formed by this novel microemulsion method can be tuned from as little as 5 to 150 nm by controlling the molar ratio of water to surfactant and the concentrations of the reagents. The confined surfactant stabilized aqueous micelles of the microemulsion allow for the preparation of nanoparticles that have a very narrow size distribution, nearly monodispersed nanoparticles having a maximum polydispersity index (volume average particle size/number average particle size) of 1.2.

In one embodiment of the method, a water-in-oil microemulsion is generated where the micelles include the soluble fluorescent dye salt and solubilized chitosan. In other embodiments of the invention, the chitosan can be replaced with other polymers containing primary amino groups, for example, polyethylenimines (PEI) or polylysine, and can be a linear polymer, a branched polymer, a hyperbranched polymer or a dendrimers. The chitosan, or other polymer, can be dissolved in a dilute acetic acid solution and mixed with ICG, generally, but not necessarily, as a disodium salt dissolved in water and mixed with a polyanionic precipitant, for example the polyacid tripolyphosphate, where the precipitant forms ammonium cations on the chitosan which form precipitating ionic cross-links and binds the ICG. A silica shell is subsequently formed about the chitosan containing core by hydrolysis and condensation of a tetraalkoxysilane, such as tetramethoxysilane or tetraethoxysilane, at the interface of the aqueous micelle containing the chitosan ICG precipitate. Other silanes that can be combined with the tetraalkoxysilane include, but are not limited to 3-mercaptopropyltrimethoxysilane, 2-methoxy(polyethyleneoxy) propyltrimethoxysilane, and N-(Trimethoxysilyl-propyl)ethyldiaminetriacetic acid trisodium salt. An aminopropyltrialkoxysilane can be included in the silane mixture to promote encapsulation of ICG and the formation of the silica shell about the chitosan ICG precipitate core and to generate sites on the nanoparticles to which moieties are attached to modify the particles for cell targeting, promotion of particle suspension, or additionally provide signals for alternate imaging techniques, such as MRI, X-ray or PAT for multimodal imaging. Metal speckles can also be deposited on the silica shell.

In another embodiment of the invention, the ICG is combined with an insoluble multivalent cation salt where, for example, a soluble barium salt and ICG are present in the micelle of a water-in-oil microemulsion, and subsequently combined with an aqueous sodium sulfate solution present in the water-in-oil microemulsion, to precipitate a Ba-ICG/BaSO₄ salt within the micelle. The barium sulfate, or other multivalent cation salt, permits formation of BaSO₄-ICG/silica core-shell nanoparticles that display CT or X-ray contrast as well as MR fluorescence traceability. The ionic interaction between a single Ba²⁺ cation and the sulfate groups of ICG is illustrated in FIG. 2. The Ba²⁺ cations and ICG dianions can be associated as the 1 to 1 ion pair shown in FIG. 2, as a 2 to 2 adduct, as any polymeric adduct, or any combinations thereof within the core-shell nanoparticles according to embodiments of the invention. The silica shell is formed about this insoluble salt core as above for the chitosan-ICG/silica core-shell nanoparticle.

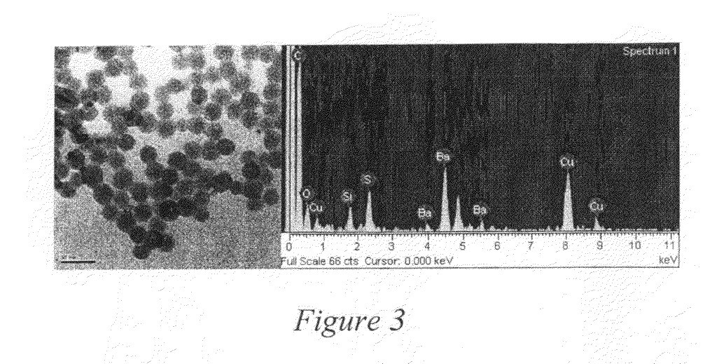

The nanoparticle cores within the micelles are coated with a silica shell to form the core-shell nanoparticle having an encapsulated dye core. Traditional sol-gel silica nanoparticle formation that one might envision to coat the core within the micelles of a microemulsion is catalyzed by NH₄OH. However it has been found that this traditional method can not be applied to the preparation of the novel core-shell nanoparticles according to embodiments of the invention because NH₄OH causes the degradation of ICG with lose of fluorescence properties during synthesis. The degradation can not be prevented by simply using a diluted NH₄OH solution. It has been discovered that by using NH₄CO₃, rather than NH₄OH, the hydrolysis and condensation of the alkoxysilanes occurs without dye degradation. For example, approximately 24 hours after introduction of the NH₄CO₃ catalyst, silica shells are formed on BaSO₄-ICG or Chitosan-ICG cores to yield the desired novel core-shell fluorescent nanoparticles. FIG. 3 shows the TEM of 20±5 nm BaSO₄-ICG silica nanoparticles.

The formation of silica nanoparticles by a sol-gel process involves two steps where hydrolysis of the precursor is followed by condensation to the nanoparticle. Using ammonium carbonate to catalyze generation of silica nanoparticles allows a high level of control over the condensation step. The use of ammonium carbonate appears to modulate the rate of silica particle formation and can affect the extent of condensation. The extent of condensation affects the mechanical and chemical stability of the nanoparticles. Hence, the nanoparticle can be formed in a manner that can be broken down (degraded) into smaller silica fragments. The particles can be effectively biodegradable, which provides significant advantageous for nanoparticles used for biological applications, such as carriers for diagnostic contrast agents, drug delivery vehicles, and other applications that employ nanoparticulates. The breakdown of the nanoparticle can be promoted by a biological environment's pH, temperature, ionic strength t, or other factors. In contrast, ammonium hydroxide catalyzed silica particle formation largely results in non-biodegradable silica particles.

In some embodiments of the invention, aminoalkysilanes, for example 3-aminopropyltrialkoxysilanes, can be included with the core material or with the tetraalkoxysilanes to enhance the ICG encapsulation efficiency. Inclusion of the amine sites in the silica matrix additionally allows for inclusion of groups for bioconjugation and targeting capability. Also, the aminoalkyl groups of the silica matrix in the shell's surface can be modified with polyethyleneglycol (PEG) or other oligomers or polymers with a strong affinity for water in some embodiments of the invention such that opsonization is prevented, allowing increased circulation times of the particles upon introduction to an organism. PEG modification can be carried out by the reaction of an N-hydroxysuccinimide ester (NHS) terminated PEG, or other reactive terminated PEG polymers, with the aminoalkyl containing silica shell.

To overcome issues associated with carrier particle inhomogeneity and allow for the facile obtainment of tunable monodispersed particle sizes of less than 100 nm, a water-in-oil microemulsion mediated synthesis strategy is carried out by modification of the process disclosed in Sharma et al., Chemistry of Materials, 2008, 20(19), 6087-94; Santra et al., Technology in Cancer Research & Treatment, 2004, 4(6), 593-602; Santra et al., Food and Bioproducts Processing, 2005, 83(C2), 136-40; Santra et al., Journal of Nanoscience and Nanotechnology, 2005, 5(6), 899-904; Santra et al., Chemical Communications, 2004, 24, 2810-1, all references incorporated herein by reference. For example, encapsulation of the surface active dye ICG in a microemulsion can be carried out as follows. Chitosan and/or a Ba²⁺ salt are dissolved in the aqueous micelles of the microemulsion, followed by addition of an ICG comprising solution such that the ICG partitions into the micelle. Subsequently a precipitant, tripolyphosphate for chitosan and/or sodium sulfate for Ba²⁺ salt, is added to cause precipitation within the micelle, entrapping ICG. Alternately, precipitation can be carried from a homogeneous aqueous solution that is subsequently used to form a microemulsion. Although many microemulsion systems can be used, encapsulation of the dyes occurs effectively in a reverse sodium bis(2-ethylhexyl)sulfosuccinate (AOT) microemulsion system, and does not occur as effectively in a common Triton X-100 (TX-100) microemulsion system. The precipitate containing micelles are then coated with silica or another metal oxide layer to encapsulate the dye. Again, a simplified schematic representation of a nanoparticle according to an embodiment of the invention is shown in FIG. 1.

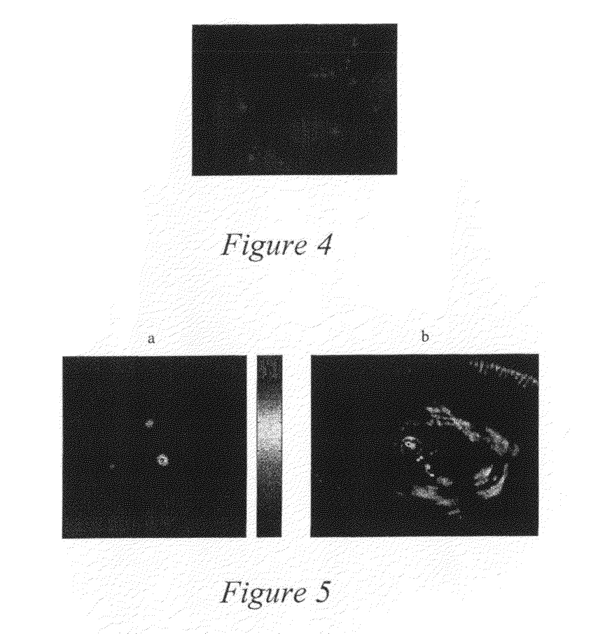

In embodiments of the invention, the novel core-shell nanoparticles containing ICG are fluorescent and are useful for imaging by fluorescence microscopy in vitro and quantitative cellular uptake by flow cytometry. For example, the nanoparticles are found to be non-toxic to cancer cells in vitro and can be taken up by cancer cells such as the breast cancer cells (BT474), as shown in the fluorescence microscopy image in FIG. 4.

Photoacoustic tomography (PAT) is an emerging powerful non-ionizing deep tissue imaging technology that offers benefits of both high optical contrast and high ultrasound resolution. PAT can image with high contrast and good spatial resolution. In PAT, NIR pulsed laser light is used to generate ultrasound waves in target structures that are detected and reconstructed for image generation. This will allow non-invasive quantization of nanoparticle contrast agent concentration inside tumors. It has been demonstrated in preliminary experiments that ICG containing nanoparticles are an excellent in vitro and in vivo photoacoustic contrast agent (FIGS. 5a and 5b).

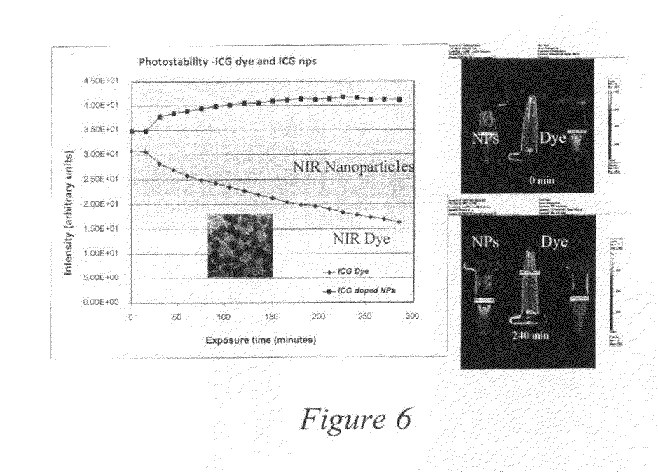

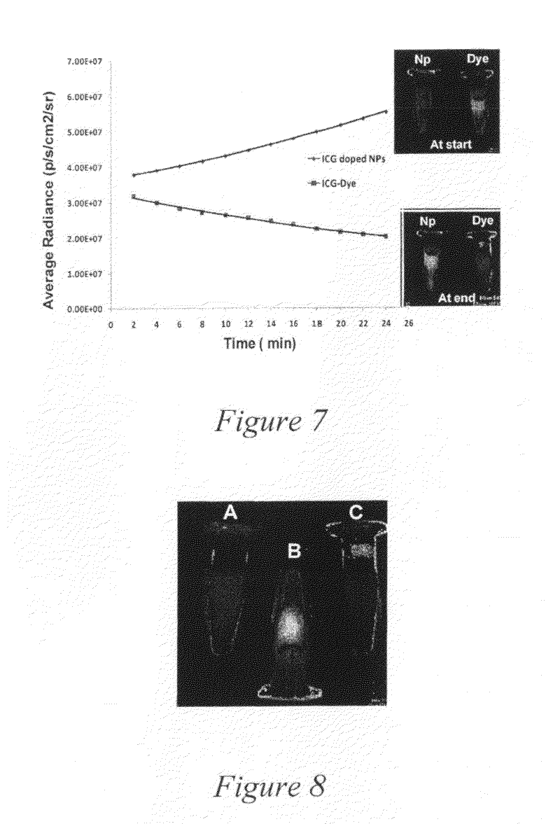

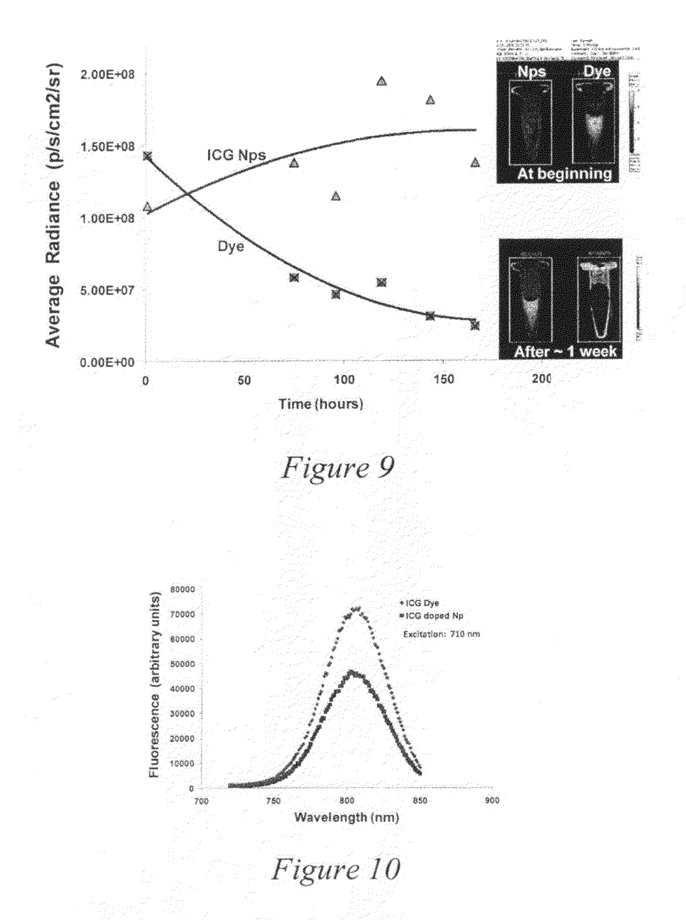

The encapsulation of ICG inside of a solid silica core significantly enhances the dyes capacity for long term imaging. FIG. 6 demonstrates that ICG-BaSO₄-aminated silica core-shell nanoparticles not only enable an improved photostability over time in comparison to the free dye, but that the intensity of fluorescence emission initially increased with time. Samples containing ICG core-shell nanoparticles and a free ICG dye solution were adjusted to display equal fluorescence emission levels. The two samples were illuminated at 710 nm for 2 minutes, held in the dark for 1 minute, and imaged and this sequence was repeated 12 times as illustrated in FIG. 7. After the 12 cycles, the exposed ICG core-shell nanoparticles were centrifuged and separated from the aqueous medium. The supernatant and the nanoparticles were imaged after resuspension in water. As shown in FIG. 8, the ICG dye leaches from the nanoparticles during photobleaching suggesting that light triggers the release of the dye from the nanoparticles to provide non-photodegraded ICG upon irradiation. The photoinduced dye release provides high fluorescence from the dye newly released from the nanoparticles that retain additional dye for release on subsequent illumination. This has therapeutic implications, allowing a controlled/triggered release of dyes from core-shell nanoparticles. The fluorescence intensity of the ICG NPs and dye was studied over 7 days (i.e., 166 hours), as shown in FIG. 9, where irradiation was carried out with only few interruptions for fluorescence measurements. As opposed to the dissolved ICG dye, the ICG doped NPs shows relatively low initial fluorescence intensity that increases through the one week period. Although not to be bound by any particular mechanism, the photostability of the ICG encapsulated in the core-shell nanoparticles is consistent with dye stabilization within the silica matrix due to inhibition of the diffusion of oxygen that promotes photodegradation into the nanoparticles, whereas slow leaching of the dye from the NPs results in the increase in fluorescence of a sample as the concentration of non-degraded dye increases with photo induced release from the core-shell nanoparticles.

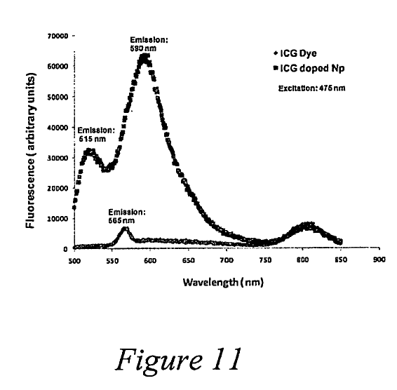

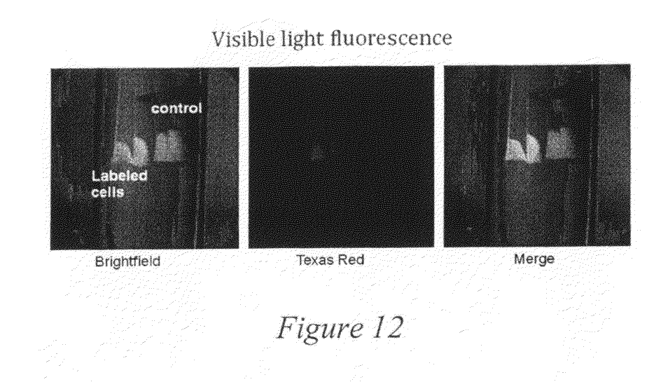

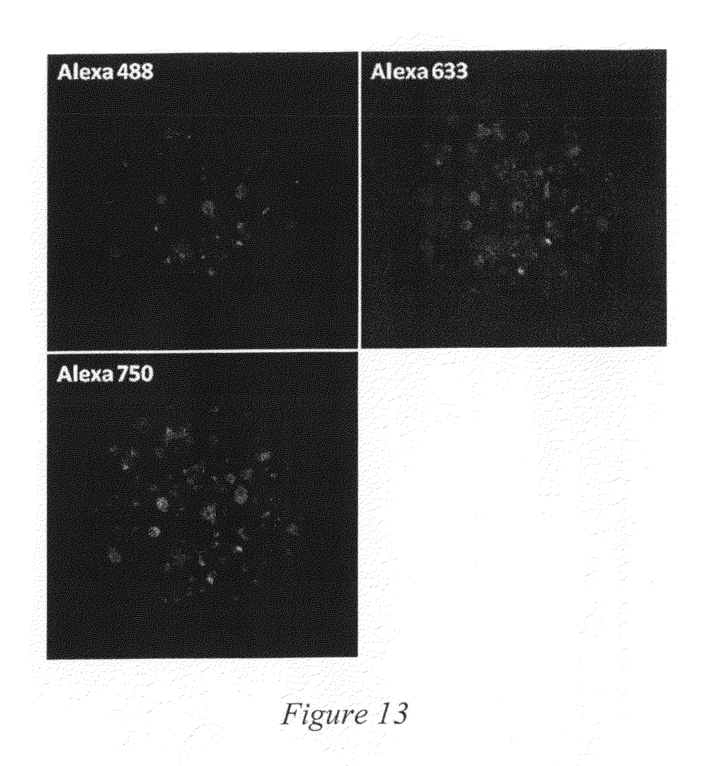

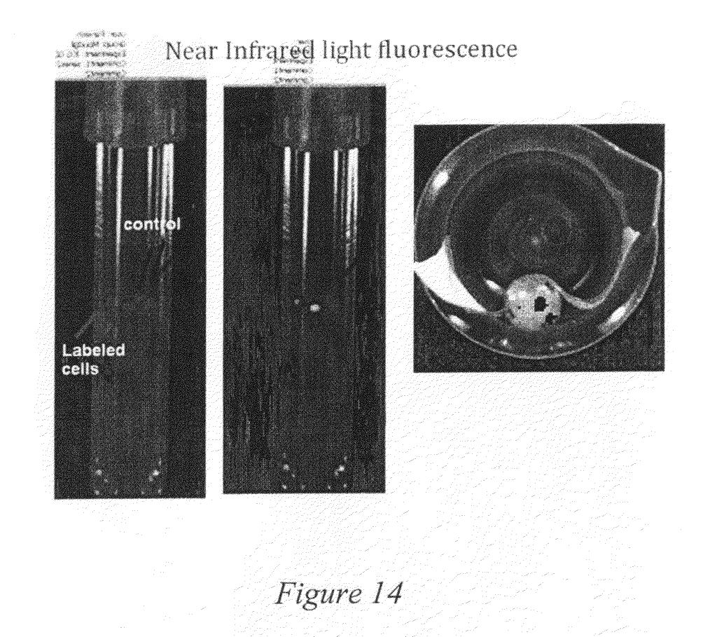

FIG. 10 shows similar maxima (805 nm) and spectral shapes, for suspended ICG core-shell nanoparticles and dissolved dye upon excitation at 710 nm. The consistency of the maxima suggests that the fluorescence property of the dye is not affected by the encapsulation process. In contrast, FIG. 11 shows the fluorescence spectra of the dye and core-shell nanoparticles upon excitation at 475 nm. Whereas the ICG dyes show two emission maxima, at 564 and 805 nm, the ICG core-shell nanoparticles show three emissions at 515 nm, 590 nm and 805 nm. These differences are suspected to arise from aggregation of the dyes within the core-shell nanoparticles. The visible emission observed at about 600 nm is advantageous for the tracking of ICG NPs with commonly available visible fluorescent microscopes. The spectral differences allow imaging of the ICG core-shell nanoparticles by visible light emission by fluorescence microscopy, as indicated in FIGS. 3, 12, and 13, as well as imaging by NIR emission as illustrated in FIGS. 13 and 14.

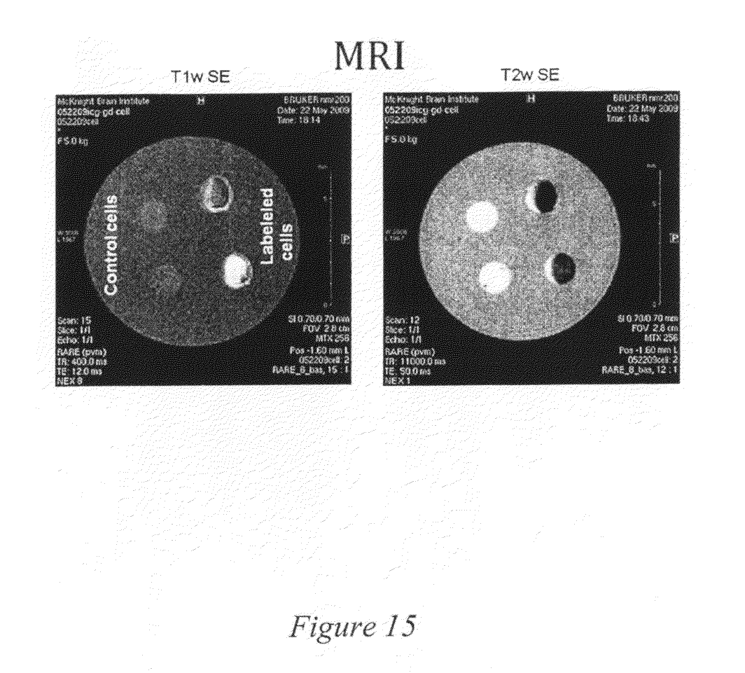

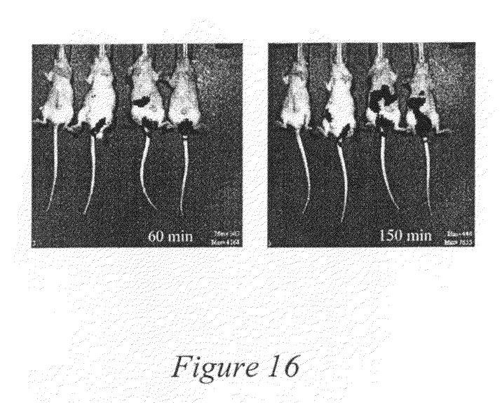

The nanoparticle synthesis can be extended to the formation of multimodal nanoparticles that can be simultaneously imaged by fluorescence and, for example, magnetic resonance imaging (MRI), in the manner disclosed in Sharma, et al., “Multimodal Nanoparticles for Non-Invasive Bio-Imaging” International Application No. PCT/US08/074,630; filed Aug. 28, 2008, and incorporated herein by reference. FIG. 15 indicates the ability of the particles to generate MR contrast using ICG-Gd core-shell nanoparticles.

The ICG core-shell nanoparticles can be use for in vivo imaging as shown in FIG. 16. In this example, 20 nm ICG core-shell nanoparticles were injected in the tail vein of the mice. As a control, one mouse (far left) was given a saline injection of similar volume. All the animals were imaged using the IVIS imaging system. As seen in FIG. 16, initially the nanoparticles are visualized in the tail vein at the site of injection and after 150 minutes they are distributed in different organs such as the liver and spleen, demonstrating that these nanoparticles can be imaged in vivo and tracked in real time. Real time imaging is useful for getting information about the pharmacokinetic distribution of the particles in vivo. Bio-conjugation with homing ligands can enable tracking accumulation of the particles in tumor region, which can be advantageous for diagnostics as well as therapeutic applications. Additionally, non-invasive real time tracking of size/surface modified nanoparticles, or cells labeled with ICG core shell particles, can be useful to understand many biological processes such as stem cell translocation.

In another embodiment of the invention, ICG core-shell nanoparticles are used therapeutically, for example, for photodynamic therapy (PDT). PDT employing ICG core-shell nanoparticles and a laser, for example a diode laser with a wavelength of 805 nm, can be used to treat: Barrett's esophagus; early esophageal cancer (adenocarcinoma or squamous cell carcinoma); obstructing esophageal cancer; persistent or recurrent esophageal cancer; gastric cancer; lung cancer; and/or macular degeneration.

All patents, patent applications, provisional applications, and publications referred to or cited herein are incorporated by reference in their entirety, including all figures and tables, to the extent they are not inconsistent with the explicit teachings of this specification.

1-34. (canceled)

35. A fluorescent core-shell nanoparticle comprising:
a core comprising a water insoluble matrix with an ionically bound fluorescent dye having at least one anionic sites; and
a shell comprising a metal oxide, wherein the nanoparticle is less than 100 nm in diameter.

36. The nanoparticle of claim 35, wherein the metal oxide comprises silicon dioxide.

37. The nanoparticle of claim 35, wherein the water insoluble matrix comprises an ionically crosslinked biocompatible polymer having cationic sites, wherein ion-pairing with the fluorescent dye ionically binds the dye within the polymer.

38. The nanoparticle of claim 35, wherein the water insoluble matrix comprises an insoluble salt of a multivalent cation wherein ion-pairing with the fluorescent dye binds the dye within the salt.

39. The nanoparticle of claim 35, wherein the water soluble fluorescent dye is indocyanine green (ICG).

40. The nanoparticle of claim 35, further comprising: a metal deposition on said shell; at least one moiety that exhibits magnetic properties; at least one moiety that exhibits paramagnetic properties; at least one moiety that exhibits X-ray opacity; a contrast agent for photoacoustic tomography (PAT) imaging; or any combination thereof.

41. The nanoparticle of claim 40, wherein the moiety that exhibits magnetic or paramagnetic properties comprises at least one lanthanide or transition metal.

42. The nanoparticle of claim 40, wherein the metal comprises gold.

43. The nanoparticle of claim 40, wherein said metal is deposited as discontinuous speckles, wherein the metal and the dielectric core have an interpenetrated gradient.

44. The nanoparticle of claim 35, further comprising at least one surface functional group.

45. The nanoparticle of claim 44, further comprising at least one biomolecule or targeting ligand attached to the surface functional group for specific targeting a tumor cell or other biological tissue.

46. The nanoparticle of claim 35, wherein the surface functional group comprising a moiety to promote suspension of the nanoparticle in water.

47. The nanoparticle of claim 46, wherein the moiety to promote suspension is derived from polyethylene glycol (PEG).

48. A method of making a fluorescent core-shell nanoparticle according to claim 35, comprising:
providing a core within the water phase of a water-in-oil microemulsion comprising an conically cross-linked biocompatible polymer having cationic sites and/or an insoluble salt of a multivalent cation and a fluorescent dye having at least one anionic sites;
adding a metal oxide precursor; and
forming a metal oxide shell by condensation of the metal oxide precursor.

49. The method of claim 48, wherein the microemulsion is a reverse sodium bis(2-ethylhexyl)sulfosuccinate (AOT) microemulsion.

50. The method of claim 48, wherein providing comprises precipitating a biocompatible polymer by a polyacid in the water phase of the microemulsion containing the dye.

51. The method of claim 48, wherein providing comprises mixing a soluble salt of the multivalent cation with a soluble salt containing an anion that combines with the multivalent cation to precipitate the insoluble salt of the multivalent cation in the water phase of the microemulsion containing the dye.

52. The method of claim 48, further comprising attaching at least one surface functional group to the shell.

53. A method of in vivo and in vitro imaging, comprising:
administering to a target a fluorescent core-shell nanoparticle according to claim 35, wherein the core comprises a water insoluble matrix with an ionically bound fluorescent dye and the shell comprises a metal oxide, wherein the nanoparticle is less than 100 nm in diameter; and
detecting a signal from the nanoparticle.

54. The method of claim 53, wherein imaging comprising fluorescence imaging alone, or in combination with one or more of X-ray, CT, and MRI imaging.

55. A therapeutic method, comprising:
administering to a target a fluorescent core-shell nanoparticle according to claim 35, wherein the core comprises a water insoluble matrix with an ionically bound fluorescent dye and the shell comprises a metal oxide, wherein the nanoparticle is less than 100 nm in diameter; and
irradiating the fluorescent core-shell nanoparticle with one or more wavelengths of electromagnetic radiation in the infrared, visible, ultraviolet, or X-ray regions of the spectrum.

56. The method of claim 33, wherein the therapy is photodynamic therapy (PDT) wherein the source or irradiation is a laser source.
